(19) United States
(12) Patent Application Publication
(10) Pub. No.: US 2005/0079557 A1
(43) Pub. Date: Apr. 14, 2005

(54) Method for the detection and/or characterisation of circulating tumour cells and the use thereof in the early diagnosis, prognosis and diagnosis of relapses and in the selection and evaluation of therapeutic treatments

(75) Inventors: Jean-Pierre Vendrell, Castelnau-Le-Lez (FR); Catherine Panabieres, Nordhouse (FR); Michel Jolivet, Saint Bonnet De Mure (FR); Genevieve Choquet-Kastylevsky, Bron (FR)

Correspondence Address: OLIFF & BERRIDGE, PLC, P.O. BOX 19928, ALEXANDRIA, VA, 22320, US

(73) Assignees: BIO MERIEUX, MARCY L'ETOILE (FR); Lapeyronie, MONTPELLIER (FR)

(21) Appl. No.: 10/505,673

(22) PCT Filed: Mar. 13, 2003
(86) PCT No.: PCT/FR03/00806

(30) Foreign Application Priority Data
Mar. 13, 2002 (FR) 02 03136

(51) Int. Cl. G01N 33/574

(52) U.S. Cl. 435/7.23

(57) ABSTRACT

The invention relates to a method for the detection and/or characterization of circulating tumour cells, in a biological sample from a patient suffering from solid cancer, which can release or secrete in vitro one or more tumour markers. The inventive method consists in: (i) depositing a known quantity of the aforementioned cells at the bottom of a culture surface to which at least one specific binding partner of the tumour marker(s) is fixed, (ii) cultivating said cells in conditions such that they release or secrete the aforementioned tumour markers which are immunocaptured at the bottom of the culture surface, (iii) eliminating the cells by washing, (iv) adding at least one specific labelled conjugate of said tumour markers, and (v) viewing the lablelling thus obtained. The invention also relates to the used of the inventive method in the early diagnosis and prognosis of the pathology, in the selection and evaluation of the effectiveness of therapeutic treatments and in the diagnosis of relapses in relation to solid cancers. Moreover, the invention related to diagnostic kits comprising a culture surface that has been coated with one or more binding partners of the specific tumour markers of the cancer being studied and the corresponding previously-labelled conjugate(s).

The present invention relates to the field of biological diagnosis in cancerology. More particularly, the present invention relates to a method for the detection and/or quantification of circulating tumor cells capable of releasing or secreting in vitro one or more tumor markers, and also to the use of this method in the early diagnosis and prognosis of the pathology, in the selection of therapeutic treatments and the evaluation of their effectiveness and in the diagnosis of relapses in relation to solid cancers.

The current diagnosis of cancers consists of a clinical diagnosis, such as breast palpation in the case of breast cancer, and/or a paraclinical examination, such as a mammogram or a scan, confirmation being carried out by means of a histological analysis such as a biopsy or surgical intervention.

Early clinical or paraclinical diagnosis of cancer is difficult, in particular due to the lack of anatomical accessibility of the cancerous regions. Consequently, many tumors are generally only detected late.

The same problem is encountered when the regions are anatomically accessible. For example, in the case of breast cancer, when a tumor is detected, in the course of a mammogram, it often has a subclinical progression of 8 years on average.

At the current time, there are no or few biological diagnostic methods which on their own enable the diagnosis of cancer.

The biological diagnostic methods currently developed make it possible to monitor the progression of an already diagnosed cancer or to screen for a relapse, for example by assaying certain tumor markers. Thus, serum markers or markers in the urine are assayed by techniques well known to those skilled in the art.

On the other hand, the direct detection of circulating tumor cells has been explored relatively little, and no routine test exists.

The possibility of detecting circulating tumor cells has been studied mainly with two diagnostic methods, namely flow cytometry and polymerase chain reaction (PCR) coupled to reverse transcription PCR (RT-PCR) (Racila E., Euhus D., Weiss A., Rao C., McConnell J., Terstappen L., Uhr J. Detection and characterization of carcinoma cells in the blood. Proc. Natl. Acad. Sci. USA 95:4589-4594 (1998), Ghossein R., Bhattacharya S., Rosai J. Molecular detection of micrometastase and circulating tumor cells in solid tumors. Clin. Cancer 5, 1950-1960 (1999) and Moss, T. J., 1991, N. Engl. J. Med., 324, 219-226). These two methods have made it possible to detect, in patients suffering from breast cancer and prostate cancer, circulating tumor cells in cells of the blood or of the bone marrow.

However, these two methods have disadvantages. In particular, neither of these methods makes it possible to quantify rare circulating cells derived from a solid tumor. In addition, it is possible to obtain false positives with PCR, such that this technique lacks sensitivity and specificity. Consequently, tumor tissue is usually needed in order to confirm the presence of tumor cells.

E. Racila et al. (1998, above) have described a method for the detection and characterization of breast cancer and prostate cancer cells in the blood, combining an immunomagnetic enrichment of epithelial cells with a flow cytometry analysis, and then, in the event of a positive response, an immunocytochemical analysis. The immunocytochemical analysis is based on the calorimetric detection of an enzyme coupled to a tumor marker (anti-cytokeratin 5, 6, 8 and 18 antibodies). This test requires a primary antibody specific for the cancer marker, a rabbit secondary immunoglobulin, an anti-alkaline phosphatase mouse immunoglobulin, alkaline phosphatase and the corresponding substrate.

The above method has the following disadvantages:
it requires specific expensive equipment, in particular for the flow cytometry analysis,
two analyses are necessary, namely a flow cytometry analysis followed by an immunocytochemical analysis, and
it lacks sensitivity.

In addition, none of the methods mentioned above makes it possible to determine the viability of the circulating cells and of their survival potential.

F. Cordoba et al. (2000, British Journal of Haematology, 108, 549-558) have described a method for the detection of myelomatous cells from patients suffering from a multiple myeloma using the known property of these cells to secrete immunoglobulin. The myelomatous cells are derived from B lymphocytes that are normally present in the blood and normally secrete immunoglobulins.

The applicant has now found, surprisingly, that the circulating tumor cells derived from solid cancers are capable of releasing or secreting certain tumor markers and that it is possible to detect this secretion.

The applicant has thus developed a novel method for the detection and/or quantification of circulating tumor cells derived from solid cancers using this particular characteristic of release or secretion of tumor markers, and overcoming the above disadvantages, namely it is simple to implement in the sense that it comprises only one analytical step and it requires no specific material. In addition, this method makes it possible to detect rare circulating cells due to its very high sensitivity and also makes it possible to determine the viability of said tumor cells. It is therefore very useful both in diagnosis and in exploration of the residual disease and in evaluation of the potential of survival, and therefore of aggressiveness, of these circulating cells.

Thus, a subject of the present invention is a method for the detection and/or quantification of circulating tumor cells, in a biological sample, which cells are capable of releasing or secreting in vitro one or more tumor markers, comprising the steps consisting in:
(i) depositing a known quantity of said cells at the bottom of a culture surface to which at least one specific binding partner of said tumor marker(s) is attached,
(ii) culturing said cells under conditions such that they release or secrete said tumor markers, which are immunocaptured at the bottom of the culture surface,
(iii) eliminating the cells by washing,
(iv) adding at least one labeled conjugate specific for said tumor markers, and
(v) visualizing the labeling thus obtained.

The method of the invention therefore makes it possible to count circulating non-hematopoietic neoplastic cells originating from biological samples from patients suffering from a solid cancer.

Solid cancers are well known to those skilled in the art. By way of example, mention may be made of breast cancer, prostate cancer, thyroid cancer, liver cancer, testicular cancer, ovarian cancer, cancer of the digestive system, lung cancer, etc.

The biological samples which may contain circulating tumor cells comprise any biological fluid, such as blood, bone marrow, effusions, milk, cerebrospinal fluid and urine.

According to a preferred embodiment, the biological samples consist of blood or bone marrow.

The tumor markers are markers specific for solid cancers, which can be released or secreted by tumor cells in vivo or in vitro under certain culture conditions.

The expression “marker released from a tumor cell” is intended to mean a membrane-bound marker which has been cleaved, and the expression “marker secreted by a tumor cell” is intended to mean both a marker secreted directly by said cell and a marker which has been cleaved in the cytoplasm and then excreted by said tumor cell.

Various antigens (protein or nonprotein) may be mentioned as a marker.

According to a preferred embodiment, said tumor markers are either membrane-bound antigens which can be released by cleavage at the bottom of the culture surface, or intracellular antigens which are secreted by said cells at the bottom of the culture surface.

By way of example of a membrane-bound antigen, mention may be made of the Muc-1 protein, which is a breast cancer cell surface protein and which is cleaved in the form of CA15-3 (carbohydrate 15-3) protein.

By way of example of a secreted antigen, mention may be made of PSA which is produced by prostate cancer cells, the cathepsin-D protein (Cath-D) which is a lysosome aspartyl protease expressed in all tissues but which is overexpressed by cancer cells in the context of breast cancer, thyroglobulin (TG) produced by cancer cells in the context of thyroid cancer, the CA 125 protein produced by cancer cells in the context of ovarian cancer, the ACE and CA 19-9 proteins produced by cancer cells in the context of colorectal cancer and the alpha-fetoprotein (AFP) produced by hepatic cells in the context of hepatocarcinomas.

According to a preferred embodiment of the invention, the tumor cells are capable of releasing as tumor marker the CA15-3 protein, and the cancer investigated is breast cancer.

According to another preferred embodiment of the invention, the tumor cells are capable of secreting as tumor marker the TG protein, and the cancer investigated is thyroid cancer.

According to another preferred embodiment of the invention, the tumor cells are capable of resecreting as tumor marker the CA 125 protein, and the cancer investigated is ovarian cancer.

According to another preferred embodiment of the invention, the tumor cells are capable of secreting as tumor marker the ACE and CA 19-9 proteins, and the cancer investigated is colorectal cancer.

According to another preferred embodiment of the invention, the tumor cells are capable of secreting as tumor marker alpha-fetoprotein, and the cancer investigated is primary liver cancer.

According to another preferred embodiment of the invention, the tumor cells are capable of secreting as tumor marker PSA, and the cancer investigated is prostate cancer.

The specific binding partners of the tumor markers consist of any partner capable of binding with the tumor markers. By way of example, mention may be made of antibodies, antibody fractions and proteins.

The binding-partner antibodies are either polyclonal antibodies or monoclonal antibodies.

The polyclonal antibodies can be obtained by immunization of an animal with at least one tumor antigen of interest, followed by recovery of the desired antibodies in purified form, by taking the serum of said animal and separating the said antibodies from the other serum constituents, in particular by affinity chromatography on a column to which is attached an antigen specifically recognized by the antibodies, in particular a tumor antigen of interest.

The monoclonal antibodies can be obtained by the hybridoma technique, the general principle of which is recalled below.

Firstly, an animal, generally a mouse (or cells in culture in the context of in vitro immunizations), is immunized with a tumor antigen of interest, for which the B lymphocytes are then capable of producing antibodies against said antigen. These antibody-producing lymphocytes are then fused with “immortal” myeloma cells (murine cells in the example) so as to give rise to hybridomas. Using the heterogeneous mixture of the cells thus obtained, a selection of the cells capable of producing a particular antibody and of multiplying indefinitely is then carried out. Each hybridoma is multiplied in the form of a clone, each resulting in the production of a monoclonal antibody whose properties of recognition with respect to the tumor antigen of interest may be tested, for example, by ELISA, by one- or two-dimensional immunoblotting, by immunofluorescence, or by means of a biosensor. The monoclonal antibodies thus selected are subsequently purified, in particular according to the affinity chromatography technique described above.

Examples of fractions of antibodies that are binding partners of the tumor markers comprise anti-CA15-3, anti-PSA, anti-alpha-fetoprotein, anti-thyroglobulin, anti-CA 19-9 and anti-CA 125 antibodies.

In the case of breast cancer, where the cells are capable of releasing the CA15-3 protein, anti-CA15-3 antibodies may be used as binding partner.

In the case of prostate cancer, where the cells are capable of secreting PSA, anti-PSA antibodies may be used as binding partner.

In the case of thyroid cancer, where the cells are capable of secreting TG, anti-TG antibodies may be used as binding partner.

In the case of ovarian cancer, where the cells are capable of secreting CA 125, anti-CA 125 antibodies may be used as binding partner.

In the case of colorectal cancer, where the cells are capable of secreting CA 19-9 or ACE, anti-CA 19-9 and anti-ACE antibodies may be used as binding partner.

In the case of hepatocarcinoma, where the cells are capable of secreting alpha-fetoprotein, anti-AFP antibodies may be used as binding partner.

The culture surface may contain several binding partners. Preferably, the culture surface contains up to four different binding partners.

According to a preferred embodiment, the culture surface contains two different types of antibodies directed against antigens specific for breast cancer, preferably anti-CA15-3 and anti-Cath-D antibodies.

The culture surface is such that it allows tumor cells to be cultured. By way of example, mention may be made of microwells, microplates, plastic surfaces and membranes.

The microwell or the microplate may itself consist of plastic such that the binding partners are attached directly to the microwell or to the microplate. They may also contain a membrane typically known to those skilled in the art, which is capable of attaching the partners of the invention. By way of example, mention may be made of nitrocellulose membranes and Immobilon-P membranes (Millipore Corporation).

The biological sample from patients of interest is deposited directly at the bottom of the culture surface, or alternatively the nonhematopoietic cells are enriched before being deposited onto said bottom.

In the case of blood samples, the cells are enriched, for example, by means of a cell separation technique on Ficoll combined with depletion of the blood cells using anti-CD45 antibodies coupled to magnetic beads (Dynal Biotech ASA, Norway). Under these conditions, a few circulating tumor cells per milliliter of total blood can be counted.

Any other method of enrichment known to those skilled in the art is suitable for the purposes of the invention.

The cells deposited onto the membrane of a microwell are counted by hemacytometry (Thomas cell, Kovas slide).

The culture conditions for the release or the secretion of the tumor markers are conventional conditions, such as 37° C. in a humid atmosphere and at 5% CO₂.

The elimination of the cells after immunocapture of the tumor markers by the binding partners attached to the bottom of the culture surface is carried out by washing consisting in using conventional washing buffers such as the PBS (phosphate buffered saline) buffer with or without bovine albumin (1%).

The conjugates, used after elimination of the cells, are conjugates typically known to those skilled in the art.

By way of example of a conjugate, mention may be made of monoclonal antibodies and polyclonal antibodies. Preferably the conjugated antibodies have a different epitope specificity than the antibodies attached to the bottom of the culture surface.

The expression “labeling the conjugates” is intended to mean the attachment of a label capable of directly or indirectly generating a detectable signal. A nonlimiting list of these labels consists of:
enzymes which produce a signal that is detectable, for example, by colorimetry, fluorescence or luminescence, such as horseradish peroxidase, alkaline phosphatase, α-galactosidase or glucose-6-phosphate dehydrogenase,
chromophores such as fluorescent, luminescent or dye compounds,
radioactive molecules such as ³²P, ³⁵S or ¹²⁵I, and
fluorescent molecules such as alexa or phycocyanins.

Indirect systems can also be used, such as, for example, ligands capable of reacting with an anti-ligand. Ligand/anti-ligand couples are well known to those skilled in the art, which is the case, for example, of the following couples: biotin/streptavidin, hapten/antibody, antigen/antibody, peptide/antibody, sugar/lectin, polynucleotide/sequence complementary to the polynucleotide. In this case, it is the ligand which carries the binding agent. The anti-ligand may be detectable directly by the labels described in the preceding paragraph or may itself be detectable by means of a ligand/anti-ligand.

These indirect detection systems can, under certain conditions, produce an amplification of the signal. This signal amplification technique is well known to those skilled in the art, and reference may be made to the prior patent applications FR98/10084 or WO-A-95/08000 by the applicant, or to the article J. Histochem. Cytochem. 45: 481-491, 1997.

According to the type of labeling of the conjugate used, those skilled in the art will add reagents for visualizing the labeling.

Thus, for example, in the case of enzymes, it is necessary to add a chromogenic substrate, such as NBT-BCPI for alkaline phosphatase or AEC for peroxidase. The addition of the chromogenic substrate then reveals a colored precipitate or immunospot (blue with NBT-BCIP and red with AEC) at the site where there was a target cell, which is a veritable protein footprint left by the cell.

All the immunospots present at the bottom of the culture surface can be visualized and counted with a binocular magnifying lens or, better still, by means of a KS ELISPOT device (company Carl Zeiss Vision GmbH) equipped with a high-performance microscope and a digital camera coupled to a computer system.

For the fluorescence labeling, all the immunospots are visualized and counted with the KS ELISPOT device adapted for a study of fluorescence.

The criteria selected for analyzing these spots include the diameter, the color, the shape, the saturation, the contrast and the diffusion gradient. In fact, the density and the granulosity of the spots decreases from the center to the periphery according to a diffusion gradient very characteristic of a protein synthesis.

When more than two binding partners are present in the culture surface, the coupling between binding partner/tumor marker is preferably revealed with secondary antibodies labeled with fluorochromes.

Thus, according to a preferred embodiment, steps (iv) and (v) of the invention are replaced with the following steps:
(iv′) adding secondary antibodies labeled with fluorochromes,
(v′) visualizing the fluorescence when there is coupling between binding partner and tumor marker.

Counting the tumor cells by means of the method of the invention makes it possible to measure their capacity for migration in solid cancers. The prognosis, the monitoring of the effectiveness of therapeutic treatments administered, the quantification of the residual disease and the diagnosis of subclinical and biological relapses in solid cancers are therefore made possible by virtue of the method of the invention, due to its very great sensitivity and specificity.

In addition, tumors can release circulating tumor cells from the very beginning of their formation, such that the method of the invention allows early diagnosis of cancer.

Consequently, another subject of the invention consists of the use of the method of the invention in the early diagnosis and the prognosis of the pathology, in the selection and evaluation of the effectiveness of therapeutic treatments, and in the diagnosis of relapses in relation to solid cancers.

According to a preferred embodiment, the method of the invention is used in the diagnosis of breast cancer, of prostate cancer, of thyroid cancer, of ovarian cancer, of colon cancer, of rectal cancer and of liver cancer.

Moreover, the method of the invention couples a method of specific detection with a method of culturing, and therefore makes it possible to verify the viable and functional nature of the tumor cells detected, this property being important in relation to the prognosis.

Another subject of the invention therefore consists of the use of the method of the invention for evaluating the survival potential of the circulating tumor cells derived from patients suffering from solid cancers.

Specifically, a positive result in the method of the invention demonstrates such a survival potential.

The method of the invention can be carried out by means of a diagnostic kit comprising a culture surface precoated with one or more binding partners of the tumor markers specific for the cancer for which it is desired to perform the investigation, and the corresponding prelabeled conjugate(s). The kit may also contain the solutions for the vigorous washing of the cells after immunocapture.

Of course, the method of the invention can be used for counting any circulating tumor cell capable of releasing or secreting at least one marker identified as a tumor marker for which a specific binding partner exists.

Thus, for example, the method of the invention makes it possible to count:
thyroid tumor cells producing thyroglobulin (TG) or calcitonin (CT),
hepatic tumor cells producing alpha-fetoproteins (AFP),
testicular tumor cells producing AFP or chorionic gonadotrophin hormone (beta-HCG),
breast tumor cells producing CA15-3, cathepsin D, PS2, Her2/neu, mammaglobin B,
ovarian tumor cells producing CA-125,
prostate tumor cells producing PSA,
tumor cells in the digestive system (colon, rectum, stomach and pancreas) producing CA19-9, CA-125, CA 19-9 and ACE, and
melanoma tumor cells producing the S100 protein.

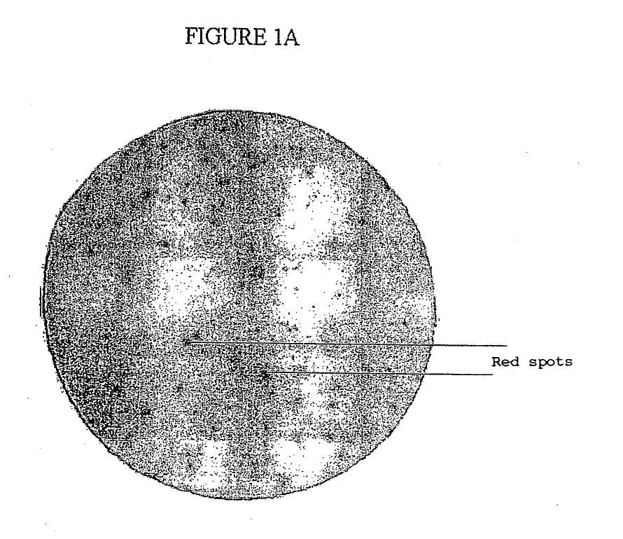

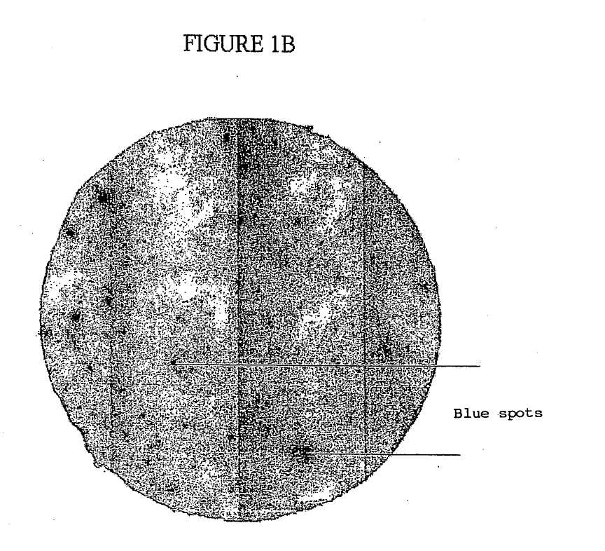

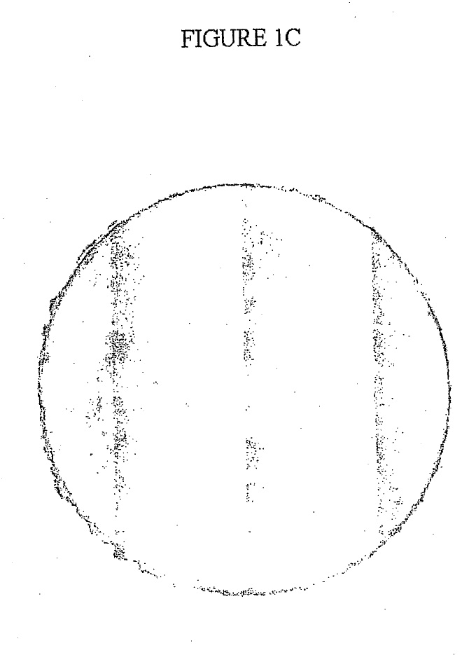

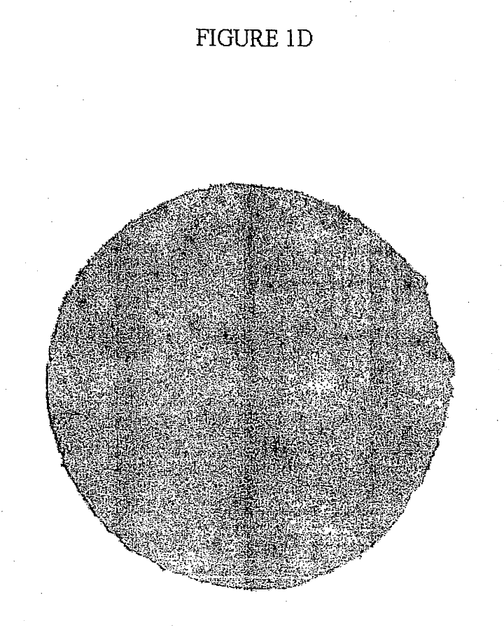

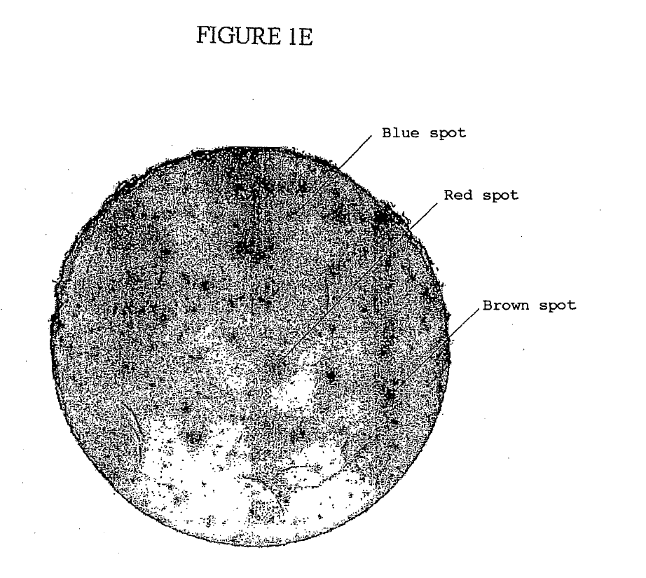

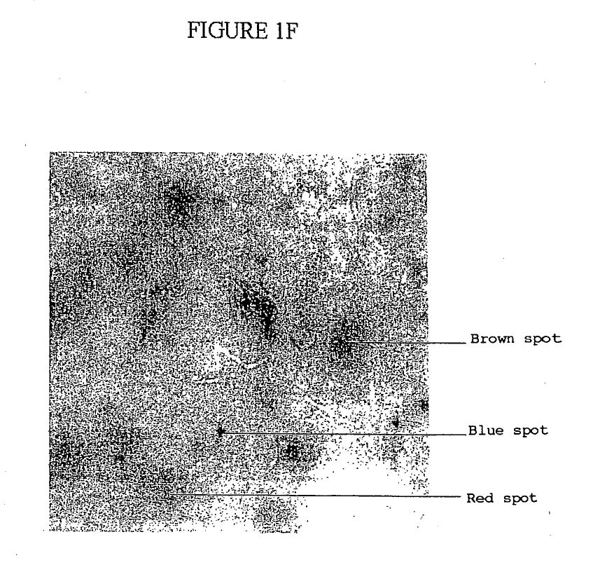

The present invention will be understood more fully by means of the following examples given only by way of nonlimiting illustration, and also by means of FIGS. 1 to 3 in the appendix, in which:

FIG. 1 (1A to 1F) shows the immunospots obtained according to the method of the invention from MCF-7 tumor cells,

FIG. 2 shows the immunospots obtained according to the method of the invention from CD45(−) cells from control individuals and from individuals suffering from metastatic breast cancer,

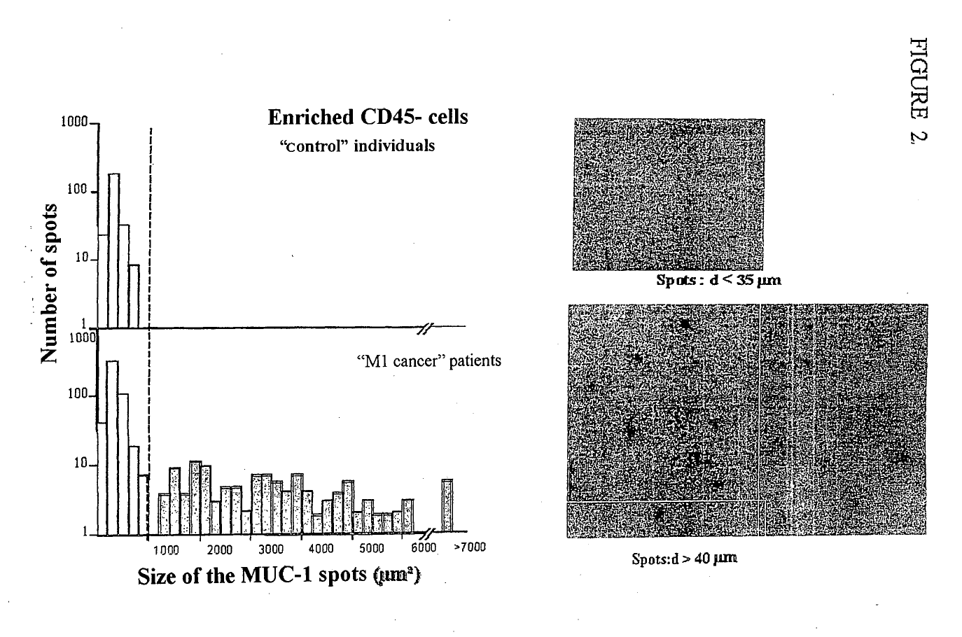

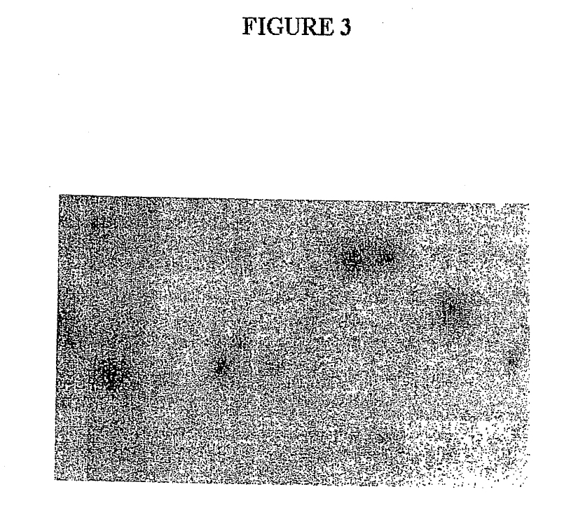

FIG. 3 shows the immunospots obtained according to the method of the invention in one individual among those suffering from metastatic breast cancer.

### EXAMPLE 1

### Counting of the Tumor Cells Originating from the MCF-7 and MDA-MB-231 (Breast Cancer) Tumor Cell Lines

The MCF-7 line was used as it secretes high levels of the Cath-D and MUC1 proteins, and the MDA-MB-231 line was used since it expresses only the Cath-D protein.

The MCF-7 and MDA-MB-231 cell lines were maintained in a Dulbecco's modified Eagle medium (DMEM, Biochrom KG, Berlin, Germany) supplemented with 1% of glutamax (Life Technologies, Paisley, Scotland), 10% of fetal calf serum (Life Technologies), 500 IU/ml of penicillin and 500 μg/ml of streptomycin (Life Technologies) in a humidified incubator containing 5% CO₂ at 37° C.

96-well microtitration plates (Nunc, Roskilde, Denmark) using an Immobilon-P membrane as solid phase (Millipore Corporation, Bedford, Mass., USA) were coated with D7E3 anti-cathepsin D monoclonal antibodies (Garcia, M., Capony, F., Derocq, D., Simon, D., Pau, B. & Rochefort, H. Characterization of monoclonal antibodies to the estrogen-regulated Mr 52,000 glycoprotein and their use in MCF7 cells. Cancer Research 45, 709-716 (1985)) and with anti-CA15-3 monoclonal antibodies (Dakocytomation, France), and were left at +4° C. overnight. The antibodies not bound to the membrane were eliminated by washing three times with PBS. The non-bound sites were then blocked with 5% bovine serum albumin (Sigma-Aldrich, St Quentin Fallavier, France) for one hour at ambient temperature.

If necessary, the cell lines were treated with 50 μg/ml of cycloheximide on a device allowing both rocking and rotation, at 37° C. for one hour, before performing the assaying.

The viable cells originating from the cell lines were counted in a hematocytometer after dye exclusion with trypan blue dye, and then serially diluted in the wells in duplicate in a growth medium at various concentrations. The plates were then incubated at 37° C. in 5% CO₂ for 24 hours.

After washing with PBS, either M1G8 anti-Cath-D monoclonal antibodies (Garcia, M., Capony, F., Derocq, D., Simon, D., Pau, B. & Rochefort, above) conjugated to horseradish peroxidase, or DF3 anti-CA15-3 monoclonal antibodies (Dakocytomation) conjugated to alkaline phosphatase (one-color process), or a mixture of these antibodies (two-color process) were added and the plates were incubated at ambient temperature.

The appropriate chromatic substrate, namely AEC staining kit (Sigma-Aldrich) for horseradish peroxidase and mixture of salt of X-phosphate/5-bromo-4-chloro-3-indolyl phosphate toluidine and of 4-nitro blue tetrazolium chloride (BCIP/NBT, Sigma), was added to each well. Red-colored (peroxidase/presence of Cath-D) or blue-colored (alkaline phosphatase/presence of CA15-3) insoluble precipitates were obtained in 5 to 10 minutes.

The plates were then washed with distilled water in order to stop the reaction.

The immunospots were counted using a KS ELISPOT device. The wells without cells or without coating of specific antibodies were included as a control.

FIG. 1 shows the results obtained. As shown in FIG. 1A-B (1A for Cath-D; 1B for CA15-3), where the one-color process was used, the use of a combination of anti-Cath-D and anti-CA15-3 monoclonal antibodies makes it possible to observe that approximately 25% of the MCF-7 cells secrete the Cath-D and/or CA15-3 proteins after 24 hours of culture in vitro. The addition of cycloheximide during the culture decreases both the size and the number of spots obtained, which confirms de novo proteic synthesis (FIG. 1C-D for Cath-D-CA15-3, respectively).

It should be noted that the cells of the MDA-MB-231 line only showed spots with the anti-Cath-D antibodies (data not shown).

Finally, the two-color technique (FIG. 1E-F) makes it possible to observe that, with the MCF-7 cell lines, approximately 17% of the spots are only “Cath-D” spots (red-colored precipitate), 82% are only “CA15-3 (MUC-1)” spots (blue-colored precipitate) and 1% are “Cath-D” and “CA15-3 (MUC-1)” double spots (brown-colored precipitate).

### EXAMPLE 2

### Detection Sensitivity of the Method of the Invention

To determine the minimum detection level of the method of the invention, serial dilutions of the cells of the MCF-7 cell line, from 100 000, 10 000, 1000, 100, 10 to 1 cell(s) per well, were used and the secretion of Cath-D was measured by the method of the invention according to the procedure described in Example 1 and by the ELISA technique (CisBioInternational, Saclay, France) in the corresponding culture supernatant.

The results are given in Table 1 below:

TABLE 1
Comparison of the sensitivity of the method of the
invention and of ELISA

| | Cath-D method of | |
|---|---|---|
| MCF-7 cells | the invention | Cath-D ELISA |
| (cells/per well) | (cells/per well) | (pmol/ml of supernatant) |
| 100 000 | ≈20 000 | 2.7 |
| 10 000 | 2350 | 0.2 |
| 1000 | 246 | 0 |
| 100 | 22 | 0 |
| 10 | 2 | 0 |
| 1 | 0.25* | 0 |

*mean of 4 wells.

As shown in the above table, with a dilution of 100 000 to 10 000 cells per well, there were so many spots that it was not possible to count them, whereas the detection of Cath-D was possible with the ELISA technique. With a dilution of 1000 cells per well, approximately 250 spots corresponding to cells secreting Cath-D (25%) were counted with the method of the invention, whereas, with the ELISA technique, no secretion of Cath-D was detected. Identical results are observed with greater dilutions.

It should be noted that, even with a single cell per well, a spot is detected with the method of the invention and that similar results were obtained with the MUC1 protein.

These data therefore show that the method of the invention has a sensitivity 10 000 times greater than that of the ELISA technique applied to the detection of a tumor marker in the culture supernatant.

### EXAMPLE 3

### Detection of Circulating Cells

Circulating epithelial cells and peripheral mononuclear cells were isolated by Ficoll-Hypaque (Pharmacia, Uppsala, Sweden) density-gradient centrifugation, from 8-10 ml of blood samples from 16 patients having a metastatic breast cancer, treated at the Centre de Recherche et de Lutte contre le Cancer [Center for Research and the Fight Against Cancer], Val d'Aurelle, Montpellier, France.

The efficiency of isolation of the epithelial cells was tested in the following way: cells of the MCF-7 cell line were diluted in blood from normal individuals, at various concentrations (1000, 100 and 10 cells per ml of blood). These aliquoted quantities of blood were then treated by means of a Ficoll-Hypaque gradient and the cells of hematological origin were eliminated by magnetic sorting using beads coated with anti-CD45 monoclonal antibodies. The CA15-3 proteins were sought in the remaining cells with the method of the invention. The efficiency of counting by means of the method of the invention was 67%, which demonstrates that the method of the invention makes it possible to recover the rare circulating cells of tumor origin.

The nonhematopoietic cells were enriched by depletion of all the CD45(+) blood cells of hematopoietic lineage originating from the peripheral mononuclear cells using anti-CD45 antibodies with magnetic labeling and a magnetic separation method according to the recommendations of Dynal Biotech ASA.

The method of the invention was carried out on the cells thus enriched from patients suffering from metastatic breast cancer and from control patients, according to the procedure described in Example 1.

The results are given in Table 2 below:

TABLE 2
Counting of Cath-D and/or MUC1 spots originating
from CD45(−) cells

| | | CD45(−) | | | |
|---|---|---|---|---|---|
| | | cells | Spots/10 ml of blood | | |
| | | (×10⁶)/10 ml | | | Cath-D + |
| Groups | Individuals | of blood | Cath-D | MUC-1* | MUC-1 |
| CI | 1 | 5 | 0 | ND | ND |
| | 2 | 1.3 | 0 | ND | ND |
| | 3 | 0.01 | ND | 0 | ND |
| | 4 | 0.01 | ND | 0 | ND |
| | 5 | 0.01 | ND | 0 | ND |
| | 6 | 0.01 | ND | 0 | ND |
| | 7 | 0.01 | ND | 0 | ND |
| | 8 | 0.01 | ND | 0 | ND |
| | 9 | 5.4 | 0 | 0 | 0 |
| | 10 | 0.2 | 0 | 0 | 0 |
| | 11 | 0.7 | 0 | 0 | 0 |
| MBC | 12 | 0.9 | 2 | 5 | 0 |
| | 13 | 1.2 | 7 | 15 | 0 |
| | 14 | 5 | 255 | 345 | 0 |
| | 15 | 2 | 12 | 250 | 0 |
| | 16 | 0.7 | 4 | 38 | 0 |
| | 17 | 1.4 | 68 | 96 | 0 |
| | 18 | 1 | 35 | 18 | 0 |
| | 19 | 1.8 | 0 | 204 | 0 |
| | 20 | 3.9 | 217 | 48 | 0 |
| | 21 | 0.9 | 16 | 39 | 0 |
| | 22 | 1.1 | 18 | 107 | 20 |
| | 23 | 0.8 | 9 | 1700 | 10 |
| | 24 | 0.6 | 20 | 25 | 0 |
| | 25 | 1.1 | 182 | 13 | 0 |
| | 26 | 1.2 | 6 | 19 | 0 |
| | 27 | 2.3 | 2 | 48 | 0 |

CI: control individuals,
MBC: metastatic breast cancer
*only the spots >1200 μ² were counted.

As indicated in the above table, when the control individuals were tested, no expression of the Cath-D protein was detected for 5/5 of the control individuals tested.

When the CA15-3 (MUC-1) spots were examined in 9 patients from the same group, numerous spots less than 1000 μm² in size were noted for numerous individuals (see FIG. 2, upper section). As regards the CA15-3 (MUC-1) spots in the patients suffering from cancer, two populations of spots were detectable, small spots less than 1000 μm² in size and larger spots, between 1200 μm² and more than 7000 μm² in size (FIG. 2, lower section), which suggests that a critical threshold of 1000 μm² could be useful for defining abnormal expression of MUC1 via CA15-3 (FIG. 2, lower section).

For the 16 patients suffering from an advanced cancer, a majority of cells expressed only a single tumor antigen, from 0.2 to 25.5 cells per ml of blood for the Cath-D protein and from 0.5 to 170 cells of blood for the CA15-3 protein, whereas only 2 patients among these 16 expressed the two proteins (FIG. 3).

For a patient investigated at the time of the first diagnosis (i.e. without treatment) of a tumor (size<2 cm), and with invasion of a sentinel lymph node, we counted 5.1 spots/ml of blood before surgical intervention and 0 spot/ml four days after.

### EXAMPLE 4

### Counting of Tumor Cells Originating from the MCF-7 and LNCAP (Prostate Cancer) Tumor Cell Lines

The LNCAP line was used since it secretes high levels of PSA protein, and the MCF-7 line was used since it does not express PSA protein.

The LNCAP cell line was maintained in an RPMI medium (Eurobio, Les Ullis, France) supplemented, QS 450 ml, with 5 ml of glutamine (2 mM), 50 ml of fetal calf serum (10%), 100 IU/ml of penicillin, 100 μg/ml of streptomycin, 2.5 ml of glucose 100 (4.5 g/l) and 5 ml of 100 mM sodium pyruvate (1 mM).

The MCF-7 cell line as described in Example 1, was used as a control.

96-well microtitration plates (Nunc, Roskilde, Denmark) using an Immobilon-P membrane as solid phase (Millipore Corporation, Bedford, Mass., USA) were coated with anti-PSA monoclonal antibodies (bioMérieux, Marcy l'Etoile, France) and were left at +4° C. overnight. The antibodies not bound to the membrane were eliminated by washing three times with PBS. The nonbound sites were then blocked with 5% bovine serum albumin (Sigma-Aldrich, St Quentin Fallavier, France) for one hour at ambient temperature.

If necessary, the cell lines were treated with 50 μg/ml of cycloheximide on a device allowing both rocking and rotation, at 37° C. for one hour, before performing the assaying.

The viable cells originating from the cell lines were counted in a hematocytometer after dye exclusion with the trypan blue dye, and then serially diluted in the wells in duplicate in a growth medium at various concentrations. The plates were then incubated at 37° C. in 5% CO₂ for 24 hours.

After washing with PBS, anti-PSA monoclonal antibodies (bioMérieux) conjugated to alkaline phosphatase (one-color process) were added and the plates were incubated at ambient temperature.

The appropriate chromatic substrate, with a mixture of salt of X-phosphate/5-bromo-4-chloro-3-indolyl phosphate toluidine and of 4-nitro blue tetrazolium chloride (BCIP/NBT, Sigma), was added to each well. Blue-colored insoluble precipitates were obtained in 5 to 10 minutes.

The plates were then washed with distilled water in order to stop the reaction.

The immunospots were counted using the KS ELISPOT device. The wells without cells or without coating of specific antibodies were included as a control.

It should be noted that the efficiency of isolation of the epithelial cells was tested as indicated in Example 3, and was 70%.

### EXAMPLE 5

### Detection of PSA-Secreting Circulating Cells

Circulating epithelial cells and peripheral mononuclear cells were isolated by Ficoll-Hypaque (Pharmacia, Uppsala, Sweden) gradient centrifugation, from 8-10 ml of blood samples from 10 patients having a metastatic prostate cancer treated at the “Beau Soleil” clinic and at the CHU [University Teaching Hospital] of Montpellier, France.

The nonhematopoietic cells were enriched by depletion of all the CD45(+) blood cells of hematopoietic lineage originating from the peripheral mononuclear cells, using anti-CD45 antibodies with magnetic labeling and a method of magnetic separation according to the recommendations of Dynal Biotech ASA.

The method of the invention was carried out on the cells thus enriched, from patients suffering from metastatic prostate cancer and from control patients, according to the procedure described in Example 1.

When the control individuals were tested, no expression of PSA protein was detected for 6/6 of the individuals tested. On the other hand, for 10 patients having bone metastases, we counted PSA-secreting circulating cells.

### EXAMPLE 6

### Counting of Tumor Cells Originating from the ML-1 (Thyroid Cancer) Tumor Cell Lines

The ML-1 line was used since it secretes high levels of the TG protein.

We were kindly provided with the ML-1 cell line by the German team of D. Grimm (Schonberger J., Bauer J. Spruss T, Weber G, Chahoud I, Eilles C, Grimm D. Establishment and characterization of the follicular thyroid carcinoma cell line ML-1, and characterization of the follicular thyroid carcinoma cell line ML-1. J. Mol. Med 2000; 78 (2): 102-10).

It was maintained in a DMEM medium (4.5 g/l glucose) supplemented with 20% of fetal calf serum, glutamine (2 mM) and sodium pyruvate (1 mM).

96-well microtitration plates (Nunc, Roskilde, Denmark) using an Immobilon-P membrane as solid phase (Millipore Corporation, Bedford, Mass., USA) were coated with anti-TG monoclonal antibodies (BioRad, Marnes la Coquette, France) and were left at +4° C. overnight. The antibodies not bound to the membrane were eliminated by washing three times with PBS. The nonbound sites were then blocked with 5% bovine serum albumin (Sigma-Aldrich, ST Quentin Fallavier, France) for one hour at ambient temperature.

The ML-1 cells were counted in a hematocytometer after dye exclusion with trypan blue dye, and then serially diluted in the wells in duplicate in a suitable growth medium at various concentrations. The plates were then incubated at 37° C. in 5% CO₂ for 24 hours.

After washing with PBS, anti-TG monoclonal antibodies (BioRad, Marnes la Coquette, France) conjugated to alkaline phosphatase (one-color process) were added and the plates were incubated at ambient temperature.

The appropriate chromatic substrate (mixture of salt of X-phosphate/5-bromo-4-chloro-3-indolyl phosphate toluidine and of 4-nitro blue-tetrazolium chloride (BCIP/NBT, Sigma) was added to each well. Blue-colored insoluble precipitates were obtained in 5 to 10 minutes.

The plates were then washed with distilled water in order to stop the reaction.

The immunospots were counted using the KS ELISPOT device. The wells without cells or without coating of specific antibodies were included as control.

It should be noted that the efficiency of isolation of the epithelial cells was tested as indicated in Example 3, and was 70%.

### EXAMPLE 7

### Detection of TG-Secreting Circulating Cells

Circulating epithelial cells and peripheral mononuclear cells were isolated by Ficoll-Hypaque (Pharmacia, Uppsala, Sweden) gradient centrifugation, from 8-10 ml of blood samples from 15 patients having a metastatic thyroid cancer, treated at the Lapeyronie hospital at the CHU [University Teaching Hospital] of Montpellier, France.

The nonhematopoietic cells were enriched by depletion of all the CD45(+) blood cells of hematopoietic lineage originating from the peripheral mononuclear cells, using anti-CD45 antibodies with magnetic labeling and a method of magnetic separation according to the recommendations of Dynal Biotech ASA.

The method of the invention was used on the cells thus enriched from patients suffering from metastatic thyroid cancer and from control patients, according to the procedure described in Example 1.

We included in our study only patients having positive TG serum levels. 15 patients were investigated, a considerable time after treatment of the primary tumor. For 5/6 patients experiencing metastatic progression, we counted TG-secreting circulating cells; for 3 patients, this cell number was increased after in vivo stimulation with thyrogen. For two patients, spots were counted without clinical complications and with normal TG levels.

### EXAMPLE 8

### Counting of Tumor Cells Originating from the BG-1 and SKOV3 (Ovarian Cancer) Tumor Cell Lines

The BG-1 line was used since it secretes high levels of the CA 125 protein, and the SKOV3 line was used since it does not express the CA 125 protein.

The BG-1 cell line was maintained in McCoy's 5a medium containing glutamine (1.5 mM) and fetal calf serum (10%).

The SKOV3 cell line was used as a control. It was maintained in a medium identical to that mentioned for the BG-1 line.

96-well microtitration plates (Nunc, Roskilde, Denmark) using an Immobilon-P membrane as solid phase (Millipore Corporation, Bedford, Mass., USA) were coated with anti-CA125 monoclonal antibodies (Dakocytomation) and were left at +4° C. overnight. The antibodies not bound to the membrane were eliminated by washing three times with PBS. The nonbound sites were then blocked with 5% bovine serum albumin (Sigma-Aldrich, ST Quentin Fallavier, France) for one hour at ambient temperature.

The BG-1 cells were counted in a hematocytometer after dye exclusion with trypan blue dye, and then serially diluted in the wells in duplicate in a suitable growth medium at various concentrations. The plates were then incubated at 37° C. in 5% CO₂ for 24 hours.

After washing with PBS, anti-CA125 monoclonal antibodies (Dakocytomation) conjugated to alkaline phosphatase (one-color process) were added and the plates were incubated at ambient temperature.

The appropriate chromatic substrate (mixture of salt of X-phosphate/5-bromo-4-chloro-3-indolyl phosphate toluidine and of 4-nitro blue tetrazolium chloride (BCIP/NBT, Sigma)) was added to each well. Blue-colored insoluble precipitates were obtained in 5 to 10 minutes.

The plates were then washed with distilled water in order to stop the reaction.

The immunospots were counted using the KS ELISPOT device. The wells without cells or without coating of specific antibodies were included as a control.

It should be noted that the efficiency of isolation of the epithelial cells was tested as indicated in Example 3, and was 70%.

### EXAMPLE 9

### Counting of Tumor Cells Originating from the Caco2 and HT-29 Tumor Cell Lines (Colorectal Cancer)

The Caco2 and HT-29 lines were used since they secrete high levels of the CA 19-9 and ACE proteins. The two cell lines were tested for the two tumor markers.

The Caco2 cell line was maintained in an MEM medium with Earle's salts and nonessential amino acids, supplemented with fetal calf serum (20%), glutamine (2 mM), sodium pyruvate (1 mM) and sodium bicarbonate (1.5 g/l).

The HT-29 cell line was maintained in McCoy's 5a medium containing glutamine (1.5 mM) and fetal calf serum (10%).

96-well microtitration plates (Nunc, Roskilde, Denmark) using an Immobilon-P membrane as solid phase (Millipore Corporation, Bedford, Mass., USA) were coated with anti-CA 19-9 (Dakocytomation) or anti-ACE (bioMérieux, Marcy l'Etoile, France) monoclonal antibodies and were left at +4° C. overnight. The antibodies not bound to the membrane were eliminated by washing three times with PBS. The nonbound sites were then blocked with 5% bovine serum albumin (Sigma-Aldrich, ST Quentin Fallavier, France) for one hour at ambient temperature.

The Caco-2 and HT-29 cells were counted in a hematocytometer after dye exclusion with trypan blue dye, and then serially diluted in the wells in duplicate in a suitable growth medium at various concentrations. The plates were then incubated at 37° C. in 5% CO₂ for 24 hours.

After washing with PBS, anti-CA 19-9 monoclonal antibodies conjugated to alkaline phosphatase or anti-ACE monoclonal antibodies conjugated to peroxidase (bioMérieux, Marcy l'Etoile, France) (one-color process) were added and the plates were incubated at ambient temperature.

The appropriate chromatic substrate for alkaline phosphatase (mixture of salt of X-phosphate/5-bromo-4-chloro-3-indolyl phosphate toluidine and of 4-nitro blue tetrazolium chloride (BCIP/NBT, Sigma)) was added to each well. Blue-colored insoluble precipitates were obtained in 5 to 10 minutes. The appropriate chromatic substrate for peroxidase, namely the AEC staining kit (Sigma-Aldrich), was added to each well. Red-colored insoluble precipitates were obtained in 10 minutes.

The plates were then washed with distilled water in order to stop the reaction.

The immunospots were counted using the KS ELISPOT device. The wells without cells or without coating of specific antibodies were included as control.

It should be noted that the efficiency of isolation of the epithelial cells was tested as indicated in Example 3, and was 70%.

### EXAMPLE 10

### Counting of Tumor Cells Originating from the Hepatic Tumor Cell Lines (Primary Liver Cancer)

The hepatic line was used since it secretes high levels of alphaprotein.

The cell line was maintained in RPMI medium containing glutamine (1.5 mM) and fetal calf serum (20%).

96-well microtitration plates (Nunc, Roskilde, Denmark) using an Immobilon-P membrane as solid phase (Millipore Corporation, Bedford, Mass., USA) were coated with anti-CA125 monoclonal antibodies (Dakocytomation) and were left at +4° C. overnight. The antibodies not bound to the membrane were eliminated by washing three times with PBS. The nonbound sites were then blocked with 5% bovine serum albumin (Sigma-Aldrich, ST Quentin Fallavier, France) for one hour at ambient temperature.

The cells were counted in a hematocytometer after dye exclusion with the trypan blue dye, and then serially diluted in the wells in duplicate in a suitable growth medium at various concentrations. The plates were then incubated at 37° C. in 5% CO₂ for 24 hours.

After washing with PBS, anti-AFP monoclonal antibodies (bioMérieux, Marcy l'Etoile, France) conjugated to peroxidase (one-color process) were added and the plates were incubated at ambient temperature.

The appropriate chromatic substrate for peroxidase, namely the AEC staining kit (Sigma-Aldrich), was added to each well. Red-colored insoluble precipitates were obtained in 10 minutes.

The plates were then washed with distilled water in order to stop the reaction.

The immunospots were counted using the KS ELISPOT device. The wells without cells or without coating of specific antibodies were included as a control.

It should be noted that the efficiency of isolation of the epithelial cells was tested as indicated in Example 3, and was 70%.

1. A method for the detection and/or quantification of circulating non hematopoietic neoplastic tumor cells, in a biological sample from a patient suffering from a solid cancer, which cells are capable of releasing or secreting in vitro one or more tumor markers, comprising the steps consisting in:
(i) depositing said sample, which cells have been counted at the bottom of a culture surface to which at least one specific binding partner of said tumor marker(s) is attached,
(ii) culturing said cells under conditions such that said non hematopoietic neoplastic tumor cells release or secrete said tumor markers, which are immunocaptured at the bottom of the culture surface,
(iii) eliminating the cells by washing,
(iv) adding at least one labeled conjugate specific for said tumor markers, and
(v) visualizing the labeling thus obtained.

2. The method as claimed in claim 1, characterized in that the biological samples consist of blood or bone marrow.

3. The method as claimed in claim 1, characterized in that said tumor markers are either membrane-bound antigens which can be released by cleavage at the bottom of the culture surface, or intracellular antigens which are secreted by said cells at the bottom of the culture surface.

4. The method as claimed in claim 1, characterized in that no more than four different binding partners, preferably no more than two partners, are attached to the bottom of the culture surface.

5. The method as claimed in claim 1, characterized in that the tumor cells are capable of secreting as tumor marker the PSA antigen, and the binding partner is an anti-PSA antibody.

6. The method as claimed in claim 1, characterized in that the tumor cells are capable of secreting as tumor marker the CA15-3 protein antigen, and the binding partner is an anti-CA15-3 antibody.

7. The method as claimed in claim 1, characterized in that the tumor cells are capable of secreting as tumor marker the TG protein antigen, and the binding partner is an anti-TG antibody.

8. The method as claimed in claim 1, characterized in that the tumor cells are capable of releasing as tumor marker the CA 125 protein antigen, and the binding partner is an anti-CA 125 antibody.

9. The method as claimed in claim 1, characterized in that the tumor cells are capable of secreting as tumor marker the ACE and/or CA 19-9 protein antigens, and the binding partners are the anti-ACE and anti-CA19-9 antibodies.

10. The method as claimed in claim 1, characterized in that the tumor cells are capable of secreting as tumor marker the alpha-fetoprotein protein antigen, and the binding partner is an anti-AFP antibody.

11. The method as claimed in claim 1, characterized in that there are two binding partners and they are preferably anti-CA15-3 and anti-Cath-D antibodies.

12. The method as claimed in claim 11, characterized in that steps (iv) and (v) are replaced with the following steps:
(iv′) adding secondary antibodies labeled with fluorochromes,
(v′) visualizing the fluorescence when there is coupling between binding partner and tumor marker.

13. The use of the method as claimed in claim 1, in the early diagnosis and the prognosis of the pathology, in the selection and evaluation of the effectiveness of therapeutic treatments, and in the diagnosis of relapses in relation to solid cancers.

14. The use as claimed in claim 13, characterized in that the cancer is breast cancer.

15. The use as claimed in claim 13, characterized in that the cancer is prostate cancer.

16. The use as claimed in claim 13, characterized in that the cancer is thyroid cancer.

17. The use as claimed in claim 13, characterized in that the cancer is ovarian cancer.

18. The use as claimed in claim 13, characterized in that the cancer is colorectal cancer.

19. The use as claimed in claim 13, characterized in that the cancer is primary liver cancer.

20. The use of the method as claimed in claim 1, for evaluating the survival potential of the circulating tumor cells derived from patients suffering from solid cancers.

21. A diagnostic kit for carrying out the method for the detection and/or quantification of circulating non hematopoietic neoplastic tumor cells, in a biological sample from a patient suffering from a solid cancer, as claimed in claim 1, comprising a culture surface precoated with one or more binding partners of the tumor markers specific for the cancer for which it is desired to perform the investigation, and the corresponding prelabeled conjugate(s).
